(19) United States
(12) Patent Application Publication
(10) Pub. No.: US 2011/0301342 A1
(43) Pub. Date: Dec. 8, 2011

(54) APPARATUS AND PROCESS FOR PURIFICATION OF PROTEINS

(75) Inventors: Chen Wang, Shrewsbury, MA (US); Robert K. Hickman, Worcester, MA (US); Edwin O. Lundell, Marlborough, MA (US); Roy D. Hegedus, Worcester, MA (US)

(73) Assignee: ABBOTT LABORATORIES, INC., Abbott Park, IL (US)

(21) Appl. No.: 13/085,630

(22) Filed: Apr. 13, 2011

Related U.S. Application Data
(60) Provisional application No. 61/345,634, filed on May 18, 2010.

(51) Int. Cl. C07K 1/36; B01D 15/10

(52) U.S. Cl. 530/413; 530/417; 530/416; 530/414; 210/198.2

(57) ABSTRACT

The invention is directed to an apparatus and method for purifying a protein. The apparatus involves the use of a capture chromatography resin, a depth filter arranged after the capture chromatography resin, and a mixed-mode chromatography resin arranged after the depth filter. The method involves providing a sample containing the protein, processing the sample through a capture chromatography resin, a depth filter, and a mixed-mode chromatography resin. A membrane adsorber or monolith may be substituted for the mixed-mode chromatography column.

### CROSS REFERENCE TO RELATED APPLICATIONS

The present application claims priority to U.S. Provisional Patent Application Ser. No. 61/345,634, filed May 18, 2010, which is incorporated herein by reference in its entirety.

### BACKGROUND OF THE INVENTION

The present invention relates generally to apparatuses for and methods of purifying proteins.

The economics of large-scale protein purification are important, particularly for therapeutic antibodies, as antibodies make up a large percentage of the therapeutic biologics on the market. In addition to their therapeutic value, monoclonal antibodies, for example, are also important tools in the diagnostic field. Numerous monoclonal antibodies have been developed and used in the diagnosis of many diseases, pregnancy, and in drug testing.

Typical purification processes involve multiple chromatography steps in order to meet purity, yield, and throughput requirements. The steps typically involve capture, intermediate purification or polish, and final polish. Affinity chromatography (Protein A or G) or ion exchange chromatography is often used as a capture step. Traditionally, the capture step is then followed by at least two other intermediate purification or polishing chromatography steps to ensure adequate purity and viral clearance. The intermediate purification or polish step is typically accomplished via affinity chromatography, ion exchange chromatography, or hydrophobic interaction, among other methods. In a traditional process, the final polish step may be accomplished via ion exchange chromatography, hydrophobic interaction chromatography, or gel filtration chromatography. These steps remove process- and product-related impurities, including host cell proteins (HCP), DNA, leached protein A, aggregates, fragments, viruses, and other small molecule impurities from the product stream and cell culture.

### SUMMARY OF THE INVENTION

Briefly, the present invention is directed to an apparatus for purifying a protein from a sample containing the protein to be purified, comprising a capture chromatography resin, a depth filter arranged with respect to the capture chromatography resin so that the sample processes through the capture chromatography resin to the depth filter, and a mixed-mode chromatography resin arranged with respect to the depth filter so that the sample processes through the depth filter to the mixed-mode chromatography resin.

Additionally, the invention is directed to a method for purifying a protein comprising providing a sample containing the protein, processing the sample through a capture chromatography resin to provide a first eluate comprising the protein, after the sample is processed through the capture chromatography resin, processing the first eluate through a depth filter to provide a filtered eluate comprising the protein, and after the first eluate is processed through the depth filter, processing the filtered eluate through a mixed-mode chromatography resin to provide a second eluate comprising the protein.

Further, the invention is directed to an apparatus and a method for purifying a protein comprising providing a sample containing the protein, processing the sample through a capture chromatography resin to provide a first eluate comprising the protein, processing the first eluate through a depth filter to provide a filtered eluate comprising the protein, and processing the filtered eluate through a membrane adsorber or a monolith to provide a second eluate comprising the protein.

### BRIEF DESCRIPTION OF THE DRAWINGS

FIG. 1 illustrates a schematic of an embodiment of the process.

FIG. 2 illustrates an alternate schematic of an embodiment of the process.

FIG. 3 illustrates an alternate schematic of an embodiment of the process.

FIG. 4 illustrates an alternate schematic of an embodiment of the process.

FIGS. 5a and 5b illustrate the HCP clearance profiles for a protein purification process.

FIGS. 6a and 6b illustrate the leached protein A clearance profiles for a protein purification process.

FIGS. 7a and 7b illustrate the aggregates clearance profiles for a protein purification process.

FIGS. 8a and 8b illustrate the DNA clearance profiles for a protein purification process.

FIGS. 9a and 9b illustrate the step yield for a protein purification process.

FIG. 10a illustrates the HCP level as a function of feed load on XOHC depth filter at different buffer conditions for a protein purification process.

FIG. 10a illustrates HCP removal by depth filtration post-Protein A capture/pH inactivation at 3000L manufacturing scale.

FIGS. 11a, 11b, and 11c illustrate impurity clearance profiles obtained via a two-column protein purification process.

FIGS. 12a and 12b illustrate the HCP clearance profiles for a protein purification process.

FIGS. 13a and 13b illustrate the leached protein A clearance profiles for a purification process.

FIGS. 14a and 14b illustrate the aggregates clearance profiles for a protein purification process.

FIGS. 15a and 15b illustrate the DNA clearance profiles for a protein purification process.

FIGS. 16a and 16b illustrate the step yield for a protein purification process.

### DETAILED DESCRIPTION OF THE EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

In an embodiment, the present invention comprises a two-chromatography step protein purification system and method. Overall recovery using the inventive system and process is acceptable and final product quality is equivalent to more traditional protocols. By eliminating specific steps in downstream processing, higher productivity is achieved while maintaining acceptable integrity and purity of the molecule. For example, minimizing the number of chromatography steps will reduce the number of process components, buffers, tanks, and miscellaneous equipment that are typically used in conventional protein purification processes.

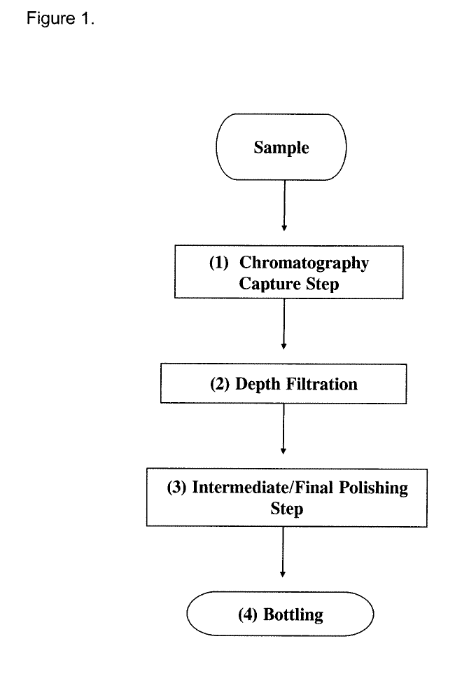

Schematic diagrams for several embodiments of the present two-chromatography step purification system are provided in FIGS. 1-4. In an embodiment of the invention, a sample which contains a protein is provided. Any sample containing a protein may be utilized in the invention. The sample, which contains a protein, may comprise, for example, cell culture or murine ascites fluid. The protein can be any protein, or fragment thereof, known in the art. In some embodiments, the protein is an antibody. In a particular embodiment, the protein is a monoclonal antibody, or fragment thereof. In some cases, the protein may be a human monoclonal antibody. In other embodiments, the protein is an immunoglobulin G antibody. In still other embodiments, the protein is a fusion protein such as an Fc-fusion protein.

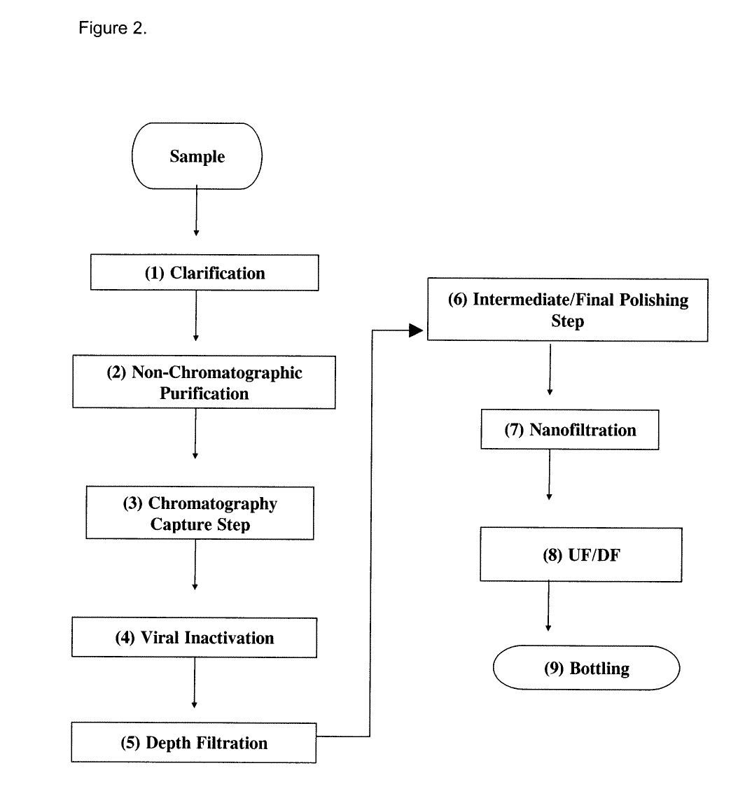

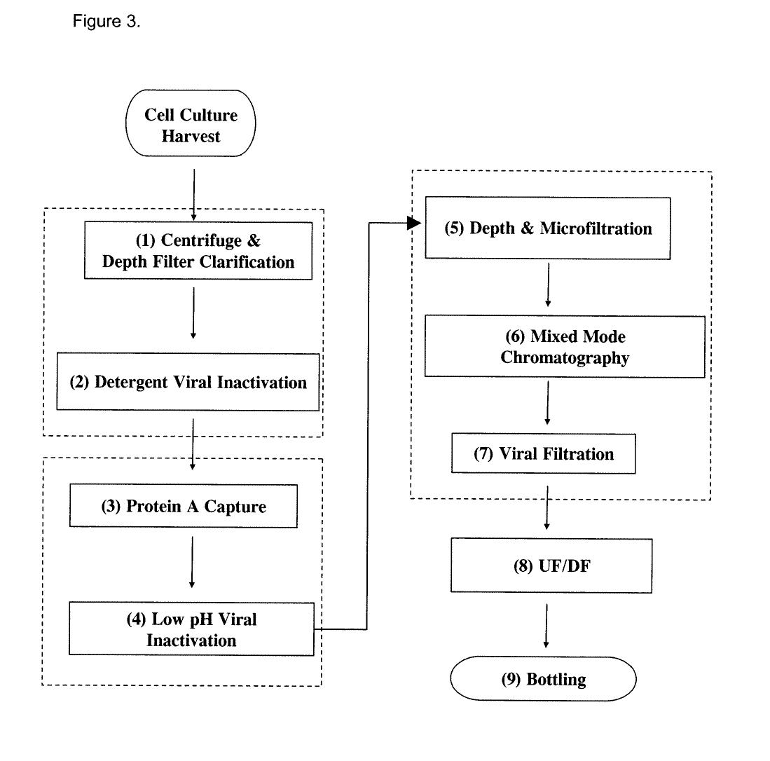

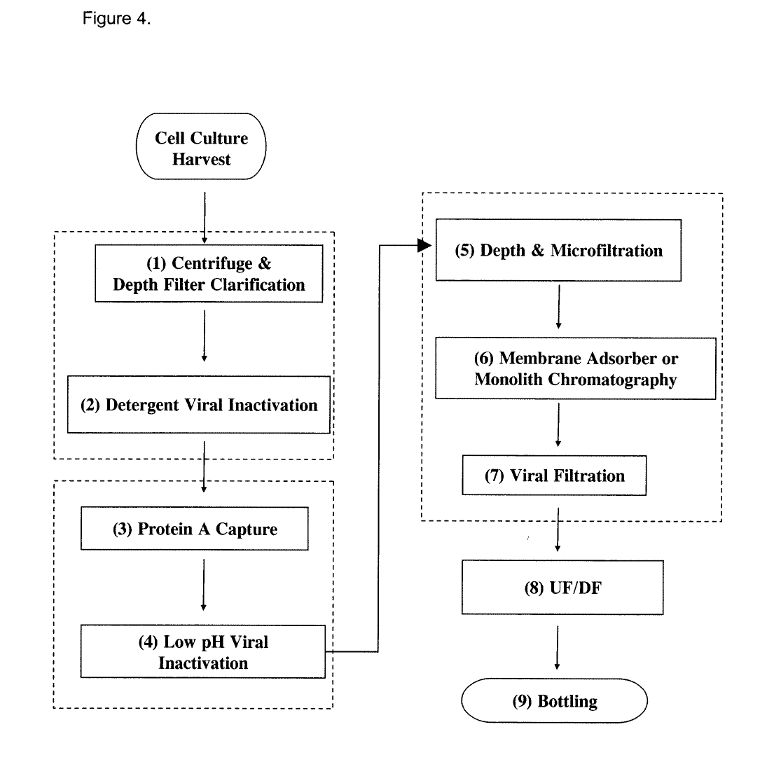

In an embodiment of the invention, the sample containing the protein may first be clarified using any method known in the art (see FIG. 2, step 1). The clarification step seeks to remove cells, cell debris, and some host cell impurities from the sample. In an embodiment, the sample may be clarified via one or more centrifugation steps (see FIGS. 3-4, step 1). Centrifugation of the sample may be performed as is known in the art. For example, centrifugation of the sample may be performed using a normalized loading of about 1×10⁻⁸ m/s and a gravitational force of about 5,000×g to about 15,000×g.

In another embodiment, the sample may be clarified via a microfiltration or ultrafiltration membrane. In some embodiments, the microfiltration or ultrafiltration membrane may be in tangential flow filtration (TFF) mode. Any TFF clarification processes known in the art may be utilized in this embodiment. TFF designates a membrane separation process in cross-flow configuration, driven by a pressure gradient, in which the membrane fractionates components of a liquid mixture as a function of particle and/or solute size and structure. In clarification, the selected membrane pore size allows some components to pass through the pores with the water while retaining the cells and cell debris above the membrane surface. In an embodiment, the TFF clarification may be conducted using, for example, a 0.1 μm or 750 kD molecular weight cutoff, 5-40 psig, and temperatures of from about 4° C. to about 60° C. with polysulfone membranes.

In yet another embodiment, the sample may be clarified via one or more depth filtration steps (see FIGS. 3-4, step 1). Depth filtration refers to a method of removing particles from solution using a series of filters, arranged in sequence, which have decreasing pore size. The depth filter three-dimensional matrix creates a maze-like path through which the sample passes. The principle retention mechanisms of depth filters rely on random adsorption and mechanical entrapment throughout the depth of the matrix. In various embodiments, the filter membranes or sheets may be wound cotton, polypropylene, rayon cellulose, fiberglass, sintered metal, porcelain, diatomaceous earth, or other known components. In certain embodiments, compositions that comprise the depth filter membranes may be chemically treated to confer an electropositive charge, i.e., a cationic charge, to enable the filter to capture negatively charged particles, such as DNA, host cell proteins, or aggregates.

Any depth filtration system available to one of skill in the art may be used in this embodiment. In a particular embodiment, the depth filtration step may be accomplished with a Millistak+® Pod depth filter system, XOHC media, available from Millipore Corporation. In another embodiment, the depth filtration step may be accomplished with a Zeta Plus™ Depth Filter, available from 3M Purification Inc.

In some embodiments, the depth filter(s) media has a nominal pore size from about 0.1 μm to about 8 μm. In other embodiments, the depth filter(s) media may have pores from about 2 μm to about 5 μm. In a particular embodiment, the depth filter(s) media may have pores from about 0.01 μm to about 1 μm. In still other embodiments, the depth filter(s) media may have pores that are greater than about 1 μm. In further embodiments the depth filter(s) media may have pores that are less than about 1 μm.

In some embodiments, the depth filtration clarification step may involve the use of two or more depth filters arranged in series. In this embodiment, for example, Millistak+® mini DOHC and XOHC filters could be arranged in series and used in the clarification step of the invention.

Any combination of these or other clarification processes which are known in the art can be utilized as the optional clarification step of the invention. For example, the clarification step may comprise both centrifugation and depth filtration (see FIGS. 3-4, step 1).

In a particular embodiment, the present system involves the use of a clarification step and a further treatment step (see FIG. 2, step 2). The further treatment step may comprise a non-chromatographic purification step.

In a particular embodiment, the further treatment step may comprise treatment with a detergent (see FIGS. 3-4, step 2). The detergent utilized may be any detergent known to be useful in protein purification processes. In an embodiment, the detergent may be applied to the sample at a low level and the sample then incubated for a sufficient period of time to inactivate enveloped mammalian viruses. The level of detergent to be applied, in an embodiment, may be from about 0 to about 1% (v/v). In another embodiment, the level of detergent to be applied may be from about 0.05% to about 0.7% (v/v). In yet another embodiment, the level of detergent to be applied may be about 0.5% (v/v). In a particular embodiment, the detergent may be polysorbate 80 (Tween® 80) or Triton® X-100. This step provides additional clearance of enveloped viruses and increases the robustness of the entire process. This step may be referred to as a detergent viral inactivation step.

In an embodiment, following the optional clarification and further purification steps of the invention, the sample may be subjected to a chromatography capture step (see FIGS. 1-2). The capture step is designed to separate the protein from the clarified sample. Often, the capture step reduces HCP, host cell DNA, and endogenous virus or virus-like particles in the sample. The chromatography mechanism utilized in this embodiment may be any mechanism known in the art to be used as a capture step. In an embodiment, the sample may be subjected to affinity chromatography, ion exchange chromatography, or hydrophobic interaction chromatography as a capture step.

In a particular embodiment of the invention, affinity chromatography may be utilized as the capture step. Affinity chromatography makes use of specific binding interactions between molecules. A particular ligand is chemically immobilized or “coupled” to a solid support. When the sample is passed over the resin, the protein in the sample, which has a specific binding affinity to the ligand, becomes bound. After other sample components are washed away, the bound protein is then stripped from the immobilized ligand and eluted, resulting in its purification from the original sample.

In this embodiment of the invention, the affinity chromatography capture step may comprise interactions between an antigen and an antibody, an enzyme and a substrate, or a receptor and a ligand. In a particular embodiment of the invention, the affinity chromatography capture step may comprise protein A chromatography, protein G chromatography, protein A/G chromatography, or protein L chromatography.

In a certain embodiment, protein A affinity chromatography may be utilized in the capture step of the invention (see FIGS. 3-4, step 3). Protein A affinity chromatography involves the use of a protein A, a bacterial protein that demonstrates specific binding to the non-antigen binding portion of many classes of immunoglobulins. The protein A resin utilized may be any protein A resin available to one in the art. In an embodiment, the protein A resin may be selected from the MabSelect™ family of resins, available from GE Healthcare Life Sciences. In another embodiment, the protein A resin may be a ProSep® Ultra Plus resin, available from Millipore Corporation. Any column available in the art may be utilized in this step. In a particular embodiment, the column may be a MabSelect™ column, available from GE Healthcare Life Sciences or a ProSep® Ultra Plus column, available from Millipore Corporation.

If protein A affinity is utilized as the chromatography step, the column may have an internal diameter of about 5 cm and a column length of about 20 cm. In other embodiments, the column length may be from about 5 cm to about 100 cm. In still another embodiment, the column length may be from about 10 cm to about 50 cm. In yet another embodiment, the column length may be about 5 cm or larger. In an embodiment, the internal diameter of the column may be from about 0.5 cm to about 2 meters. In another embodiment, the internal diameter of the column may be from about 1 cm to about 10 cm. In still another embodiment, the internal diameter of the column may be about 0.5 cm or larger.

The specific methods used for the chromatography capture step, including flowing the sample through the column, wash, and elution, depend on the specific column and resin used and are typically supplied by the manufacturers or are known in the art. As used herein, the term “processed” may describe the process of flowing or passing a sample through a chromatography column, resin, membrane, filter, or other mechanism, and shall include a continuous flow through each mechanism as well as a flow that is paused or stopped between each mechanism.

Following the chromatography capture step, the eluate may be subjected to viral inactivation (see FIGS. 2-4, step 4). In an embodiment, this viral inactivation step may comprise low-pH viral inactivation (see FIGS. 3-4, step 4). In one aspect, use of a high concentration glycine buffer at low pH for elution may be employed, without further pH adjustment, in a final eluate pool in the targeted range for low-pH viral inactivation. Alternatively, acetate or citrate buffers may be used for elution and the eluate pool may then be titrated to the proper pH range for low-pH viral inactivation. In an embodiment, the pH is from about 2.5 to about 4. In a further embodiment, the pH is from about 3 to about 4.

In an embodiment, once the pH of the eluate pool is lowered, the pool is incubated for a length of time from about 15 to about 90 minutes. In a particular embodiment, the low-pH viral inactivation step may be accomplished via titration with 0.5 M phosphoric acid to obtain a pH of about 3.5 and the sample may then be incubated for 1 hour.

After the low-pH viral inactivation step, the inactivated eluate pool may be neutralized to a higher pH. In an embodiment, the neutralized, higher pH may be a pH of from about 6 to about 10. In another embodiment, the neutralized, higher pH may be a pH of from about 8 to about 10. In yet another embodiment, the neutralized, higher pH may be a pH of from about 6 to about 10. In yet another embodiment, the neutralized, higher pH may be a pH of from about 6 to about 8. In yet another embodiment, the neutralized, higher pH may be a pH of about 8.1.

In an embodiment, the pH neutralization may be accomplished using 1 M Tris pH 9.5 buffer or another buffer known in the art. The conductivity of the inactivated eluate pool may then be adjusted with purified or deionized water. In an embodiment, the conductivity of the inactivated eluate pool may be adjusted to from about 0.5 to about 50 mS/cm. In another embodiment, conductivity of the inactivated eluate pool may be adjusted to from about 6 to about 8 mS/cm.

In alternative embodiments, the viral inactivation step may be carried out using other methods known in the art. For example, the viral inactivation step may comprise, in various embodiments, treatment with acid, detergent, chemicals, nucleic acid cross-linking agents, ultraviolet light, gamma radiation, heat, or any other process known in the art to be useful for this purpose.

Following the optional viral inactivation step, the inactivated eluate pool may be subjected to depth filtration, as described above (see FIGS. 1-4). This depth filtration step may be in addition to the use of depth filtration as a clarification step. In an embodiment, this step may involve the use of two or more depth filters arranged in series. With appropriate sizing of the depth filter, based upon the processing conditions known in the art, various impurities can be removed or reduced from the process stream before further processing.

In an embodiment, the depth filtration step may be followed by or combined with a sterile filtration step (see FIGS. 3-4, step 5). Any sterile filter known in the art may be useful in this embodiment. In an embodiment, the sterile filter is a microfilter. In one aspect of the invention, the sterile filter may comprise a Sartopore® 2 sterilizing grade filter. The sterilizing filter, for example, may have a 0.45 μm pre-filter in front of a 0.2 μm final filter. In another embodiment, the sterilizing filter may have membrane pores that are from about 0.1 μm to about 0.5 μm. In other embodiments, the sterilizing filter may have membrane pores that are from about 0.1 μm to about 0.3 μm. In one aspect, the sterilizing filter may have membrane pores that are about 0.22 μm. In an embodiment, the sterilizing filter may be arranged in series with the depth filter.

Following depth filtration and optional sterile filtration, the sample may then be subjected to an intermediate/final polishing step (see FIGS. 1-2). In an embodiment, the intermediate/final polishing step may comprise a mixed-mode (also known as multimodal) chromatography step (see FIG. 3, step 6). In this step, the residual HCP, DNA, leached protein A, and aggregates are cleared from the sample. The mixed-mode chromatography step utilized in this invention may utilize any mixed-mode chromatography process known in the art. Mixed mode chromatography involves the use of solid phase chromatographic supports in resin, monolith, or membrane format that employ multiple chemical mechanisms to adsorb proteins or other solutes. Examples useful in the invention include, but are not limited to, chromatographic supports that exploit combinations of two or more of the following mechanisms: anion exchange, cation exchange, hydrophobic interaction, hydrophilic interaction, thiophilic interaction, hydrogen bonding, pi-pi bonding, and metal affinity. In particular embodiments, the mixed-mode chromatography process combines: (1) anion exchange and hydrophobic interaction technologies; (2) cation exchange and hydrophobic interaction technologies; and/or (3) electrostatic and hydrophobic interaction technologies.

In an embodiment, the mixed-mode chromatography step may be accomplished by using a column and resin such as the Capto® adhere column and resin, available from GE Healthcare Life Sciences. The Capto® adhere column is a multimodal medium for intermediate purification and polishing of monoclonal antibodies after capture. In a particular embodiment, the mixed-mode chromatography step may be conducted in flow-through mode. In other embodiments, the mixed-mode chromatography step may be conducted in bind-elute mode.

In other embodiments, the mixed-mode chromatography step may be accomplished by using one or more of the following systems: Capto® MMC (available from GE Healthcare Life Sciences), HEA HyperCel™ (available from Pall Corporation), PPA HyperCel™ (available from Pall Corporation), MBI HyperCel™ (available from Pall Corporation), MEP HyperCel™ (available from Pall Corporation), Blue Trisacryl M (available from Pall Corporation), CFT™ Ceramic Fluoroapatite (available from Bio-Rad Laboratories, Inc.), CHT™ Ceramic Hydroxyapatite (available from Bio-Rad Laboratories, Inc.), and/or ABx (available from J. T. Baker). The specific methods used for the mixed-mode chromatography step may depend on the specific column and resin utilized, and are typically supplied by the manufacturer or are known in the art.

Each column utilized in the process may be large enough to provide maximum throughput capacity and economies of scale. For example, in certain embodiments, each column can define an interior volume of from about 1 L to about 1500 L, of from about 1 L to about 1000 L, of from about 1 L to about 500 L, or of from about 1 L to about 250 L. In some embodiments, the mixed-mode column may have an internal diameter of about 1 cm and a column length of about 7 cm. In other embodiments, the internal diameter of the mixed-mode column may be from about 0.1 cm to about 10 cm, from about 0.5 cm to about 5 cm, from about 0.5 cm to about 1.5 cm, or may be about 1 cm. In an embodiment, the column length of the mixed-mode column may be from about 1 to about 50 cm, from about 1 to about 20 cm, from about 5 to about 10 cm, or may be about 7 cm.

In some embodiments, the inventive systems are capable of handling high titer concentrations, for example, concentrations of about 5 g/L, about 6 g/L, about 7 g/L, about 8 g/L, about 9 g/L, about 10 g/L, about 12.5 g/L, about 15 g/L, about 20 g/L, about 25 g/L, concentrations of from about 1 g/L to about 5 g/L, concentrations of from about 5 g/L to about 10 g/L, concentrations of from about 5 g/L to about 12.5 g/L, concentrations of from about 5 g/L to about 15 g/L, concentrations of from about 5 g/L to about 20 g/L, or concentrations of from about 5 g/L to about 55 g/L, or concentrations of from about 5 g/L to about 100 g/L. For example, some of the systems are capable of handling high antibody concentrations and, at the same time, processing from about 200 L to about 2000 L culture per hour, from about 400 L culture to about 2000 L per hour, from about 600 L to about 1500 L culture per hour, from about 800 L to about 1200 L culture per hour, or greater than about 1500 L culture per hour.

In an embodiment of the invention, shown in FIG. 3, the capture column and mixed mode column are the only chromatography columns utilized. In one aspect of the present embodiment, no third chromatography column is employed; however, should further processing require additional chromatography steps, those steps are also encompassed herein.

In an embodiment, the intermediate/final polish step may be accomplished via one or more membrane adsorbers or monoliths rather (see FIG. 4, step 6) than a mixed-mode column. Membrane adsorbers are thin, synthetic, microporous or macroporous membranes that are derivatized with functional groups akin to those on the equivalent resins. On their surfaces, membrane adsorbers carry functional groups, ligands, interwoven fibers, or reactants capable of interacting with at least one substance in contact with in a fluid phase, moving through the membrane by gravity. The membranes are typically stacked 5 to 15 layers deep in a comparatively small cartridge, generating a much smaller footprint than columns with a similar output. The membrane adsorber utilized herein may be a membrane ion-exchanger, mixed-mode, ligand membrane and/or hydrophobic membrane.

In an embodiment, the membrane adsorber utilized may be ChromaSorb™ Membrane Adsorber, available from Millipore Corporation. ChromaSorb™ Membrane Adsorber is a membrane-based anion exchanger designed for the removal of trace impurities including HCP, DNA, endotoxins, and viruses for MAb and protein purification. Other membrane adsorbers that could be utilized include Sartobind® Q (available from Sartorium BBI Systems GmbH), Sartobind® S (available from Sartorium BBI Systems GmbH), Sartobind® C (available from Sartorium BBI Systems GmbH), Sartobind® D (available from Sartorium BBI Systems GmbH), Pall Mustang™ (available from Pall Corporation), or any other membrane adsorber known in the art.

As set forth above, monoliths may alternatively be utilized in the intermediate/final polishing step of the invention (see FIG. 4, step 6). Monoliths are one-piece porous structures of uninterrupted and interconnected channels of specific controlled size. Samples are transported through monoliths via convection, leading to fast mass transfer between the mobile and stationary phase. Consequently, chromatographic characteristics are non-flow dependent. Monoliths also exhibit low backpressure, even at high flow rates, significantly decreasing purification time. In an embodiment, the monolith may be an ion-exchange or mixed-mode ligand-based monolith. In one aspect, the monolith utilized may include UNO monoliths (available from Bio-Rad Laboratories, Inc.) or ProSwift or IonSwift monoliths (available from Dionex Corporation).

In still another embodiment, the intermediate/final polish step may be accomplished via an additional depth filtration step rather than membrane adsorbers, monoliths, or a mixed-mode column. In this embodiment, the depth filtration utilized for intermediate/final polish may be a CUNO VR depth filter. In this embodiment, the depth filter may serve the purpose of intermediate/final polish as well as viral clearance.

Following the intermediate/final polish or mixed-mode chromatography step, the eluate pool may be subjected to a viral or nanofiltration step (see FIGS. 2-4, step 7). In an embodiment, this filtration step is accomplished via a nanofilter or viral filter. As shown in FIGS. 2-4, step 8, the viral or nanofiltration step may be optionally followed by UF/DF, to achieve the targeted drug substance concentration and buffer condition before bottling. The viral filtration and UF/DF steps can be combined or replaced by any process(es) known in the art known to provide a purified protein that is acceptable for bottling (FIGS. 2-4, step 9).

As will be seen, the inventive process can provide consistently high product quality and process yield. In addition, compared to the traditional protein purification processes, the inventive process may reduce the total downstream batch processing time by about 40% to 50% and significantly reduce production cost.

In an embodiment, the entire purification process can be completed in less time than what is typical, for example, the entire process can be accomplished in less than five days. For example, steps 1 and 2, or steps 3 and 4, or steps 5, 6 and 7 (as shown in broken lines in FIGS. 3-4), respectively, can be completed within a day or less. This is approximately one half of the purification time needed for a typical three-column process.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples.

### Example 1

Purification experiments were carried out and compared with a standard three-column process for yield and purity. A clarified harvest (herein designated “CH”) for MAb A and a protein A eluate (herein designated “PAE1”) of MAb B were used in this study. Two runs of each protein sample were conducted (Case 1 and Case 2).

### Procedures

The samples were centrifuged and filtered using Millistak+® Pod depth filter system, XOHC media, available from Millipore Corporation. After filtration, Tween® 80 at 0.5% (v/v) final concentration was added to the clarified harvest and the mixture was chilled with ice packs. A 5 cm (internal diameter (i.d.))×20 cm (column length) ProSep® Ultra Plus column was used for capture. After equilibration, the column was loaded with CH of MAb A to 45 g/L at 400 cm/hr, followed by washes with equilibration and intermediate salt buffers and then eluted with pH 3.5 acetate buffer. The column was regenerated using 0.15 M phosphoric acid before the next run. The eluate pool was then mixed and titrated with 0.5 M phosphoric acid to pH 3.5, incubated for 1 hour and then neutralized to pH 8.1 using 1 M Tris, pH 9.5 buffer. The conductivity of the pool was adjusted to 6-8 mS/cm using Milli-Q® water.

Two sets of conditions were evaluated for the subsequent steps. In one case, the pH-inactivated protein A pool was filtered through a 23 cm² Millistak+® mini XOHC filter at a load of 60 L/m² followed by a 13 cm² 0.45/0.22 μm Sartopore® 2 membrane filter, available from Sartorius Stedim Biotech. In the second case, two Millistak+® mini XOHC filters were connected in series and loaded with protein A eluate pool at 100 L/m² per device. Each filtrate was then flowed through either: (1) a 1 cm (i.d.)×7 cm Capto® adhere column; or (2) in a standard, three-column process that includes a 0.66 cm (i.d.)×21.3 cm Q Sepharose® Fast Flow (QSFF) column (available from GE Healthcare Life Sciences) followed by bind-elute purification on a 0.66 cm (i.d.)×15.2 cm Phenyl Sepharose® HP column (available from GE Healthcare Life Sciences). The detailed fine purification conditions are summarized in Table 1. All steps were operated at room temperature.

TABLE 1
Experimental conditions for each polishing chromatography step.

| Polishing | | | | | | Pooling |
|---|---|---|---|---|---|---|
| process | Load | Equilibration | Wash | Elution | Cleaning | criteria |
| Capto ® | pH 8.1, 6-8 | 25 mM Tris, | 25 mM Tris, | 25 mM Tris, | 1M | 200 mAU |
| adhere flow- | mS/cm, 180- | pH 8.1, ~6 | pH 8.1, ~6 | pH 8.1, 1M | NaOH, 5 | at load to |
| through | 195 mg/ml | mS/cm, 5 CV, | mS/cm, 20 | NaCl, 5 CV, 1 | CV, 1 | 200 mAU |
| | load, 3 min | 3 min RT | CV, 3 min RT | min RT | min RT | at wash |
| | RT | | | | | |
| Q | pH 8, 6 | 25 mM Tris, | 25 mM Tris, | 25 mM | 0.5M | 200 mAU |
| Sepharose ® | mS/cm, 80 | pH 8, ~6 | pH 8, ~6 | Sodium | NaOH, 3 | at load to |
| Fast Flow | mg/ml load, | mS/cm, 8.5 | mS/cm, 5 CV, | Phosphate, | CV, 17 | 200 mAU |
| flow-through | 12.8 min RT | min RT, 5 CV | 12.8 min RT | 1M NaCl, pH | min RT | at wash |
| | | | | 7, 5 CV, 17 | | |
| | | | | min RT | | |
| Phenyl | 20 mM | 20 mM | 25 mM | 11 mM | Water, 4 | 200 mAU |
| Sepharose ® | Sodium | Sodium | Sodium | Sodium | CV, 24 | to 200 |
| HP | Phosphate, | Phosphate, | Phosphate, | Phosphate, | min RT; | mAU |
| bind-elute | 1.1M | 1.1M | 1.4M | 0.625M | 1M | during |
| | ammonium | ammonium | ammonium | ammonium | NaOH, 3 | elution |
| | sulfate, pH | sulfate, pH | sulfate, pH | sulfate, pH | CV, 24 | |
| | 7.0, 64 | 7.0, 5 CV, | 7.0, 5 CV, | 7.0, 5 CV, 24 | min RT | |
| | mg/ml load, | 15.2 min RT | 15.2 min RT | min RT | | |
| | 15.2 min RT | | | | | |

* RT—flow residence time

Similar experiments were carried out to purify PAE1 for MAb B. Instead of starting from the clarified harvest, the protein A eluate pool sample was used in this case. The XOHC depth filter was loaded to 60 L/m² and the Capto® adhere column was loaded to 200 to 250 g/L in two runs. Key impurities such as HCP, leached protein A, aggregates/fragments and DNA, as well as step yield were measured for each step.

### Results

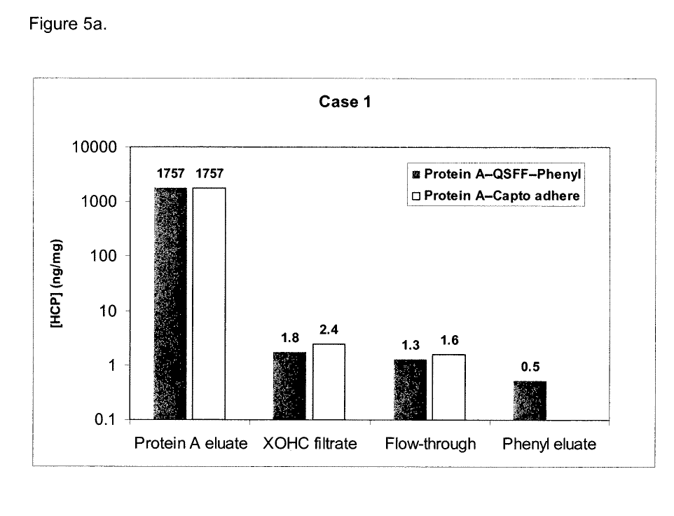

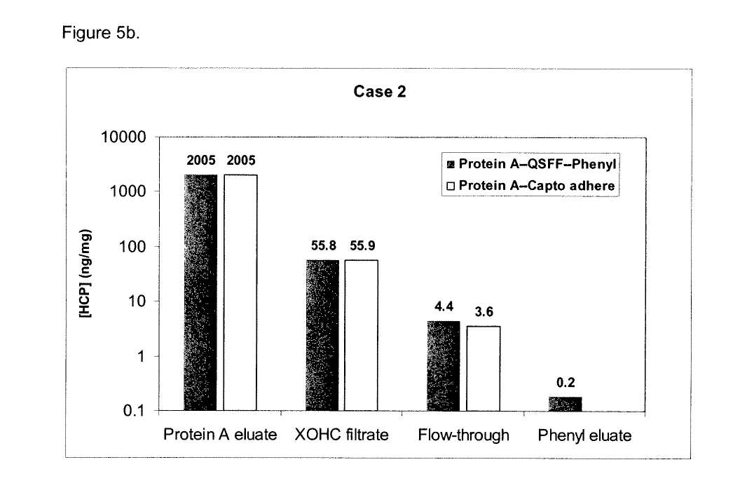

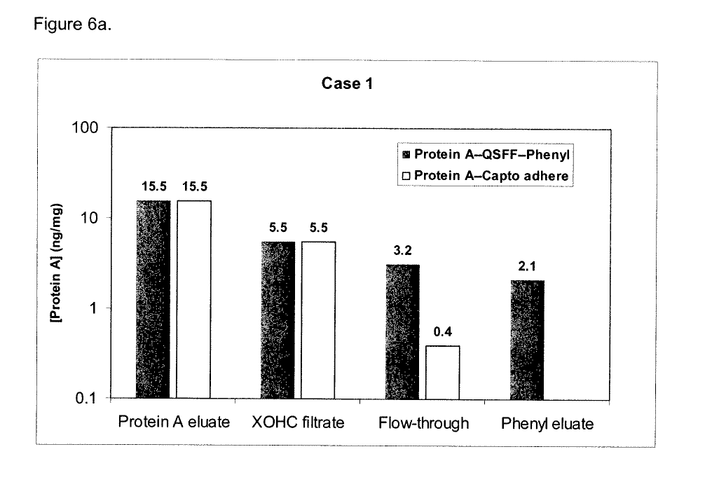

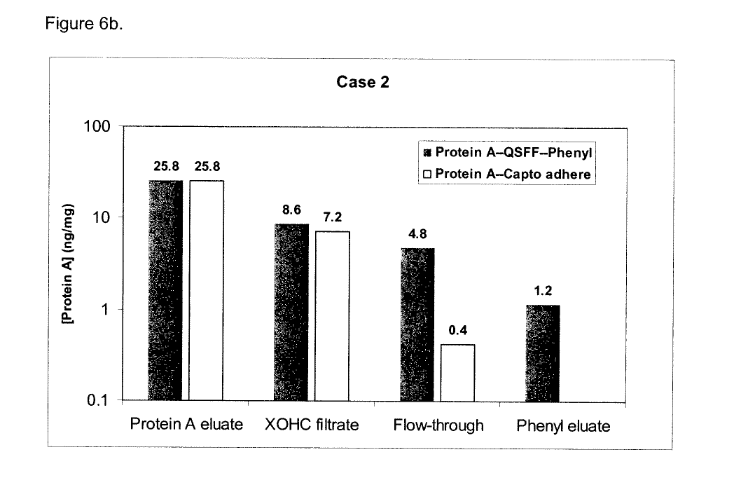

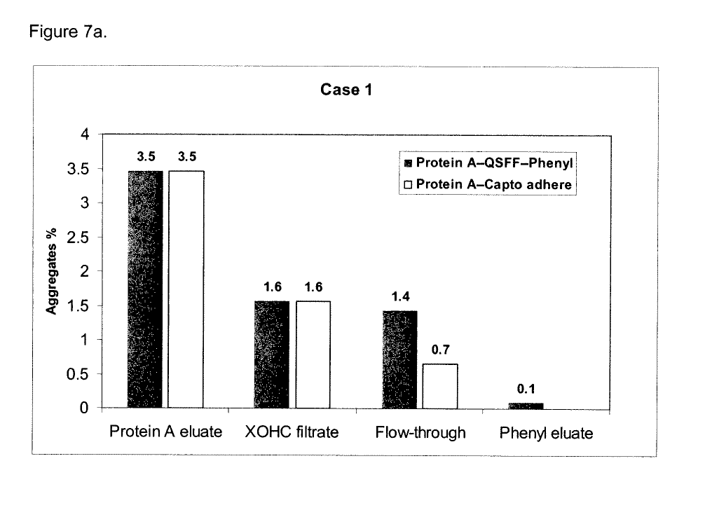

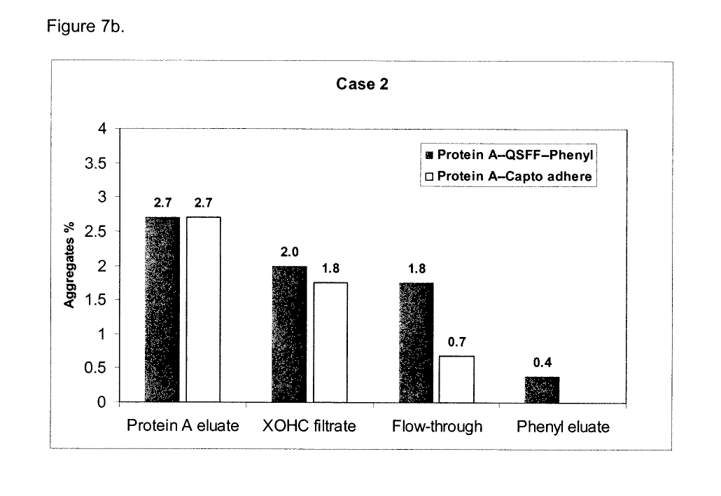

FIGS. 5-8 show the levels of HCP, leached protein A, aggregates, and DNA after each unit operation for a three-column process (labeled as Protein A-QSFF-Phenyl) versus the present two-column process (labeled as Protein A-Capto adhere). As can be seen, the protein A eluate pool (labeled as Protein A eluate) contained about 1700 to 2000 ng/mg HCP, 15 to 26 ng/mg leached protein A, and 2.7% to 3.5% high molecular weight species (DNA was not assayed in this case). After low pH inactivation, the protein A eluate was filtered through an XOHC depth filter at two different loading levels.

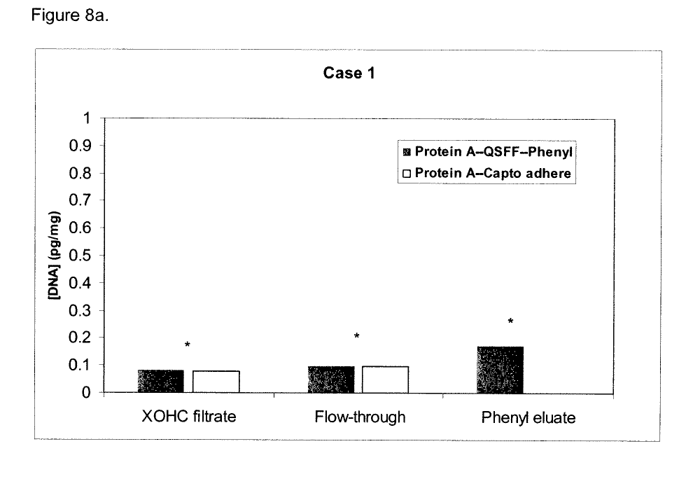

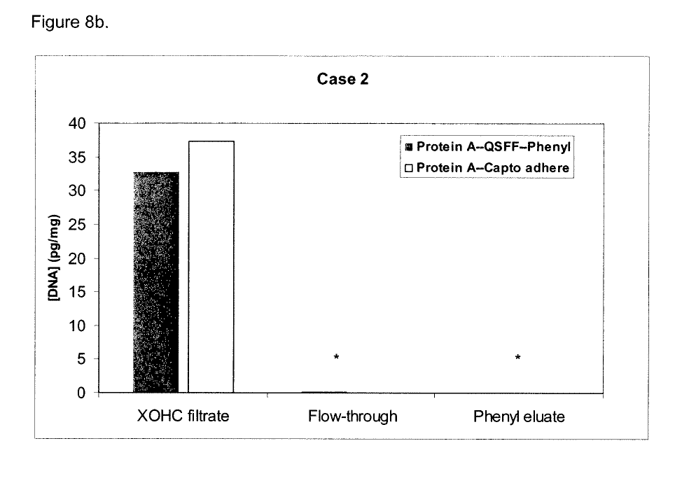

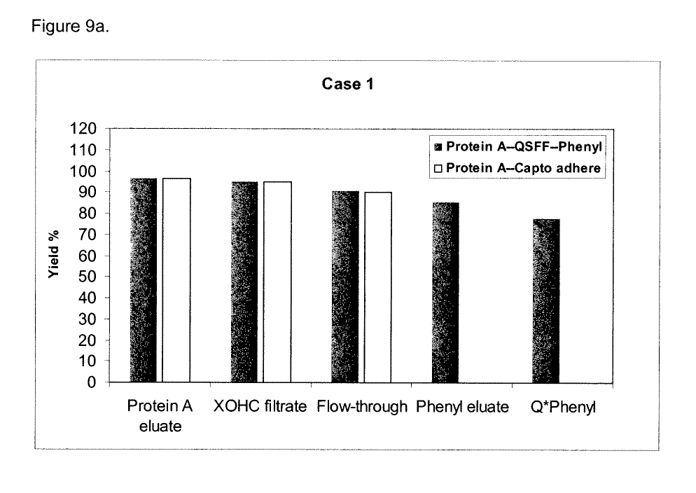

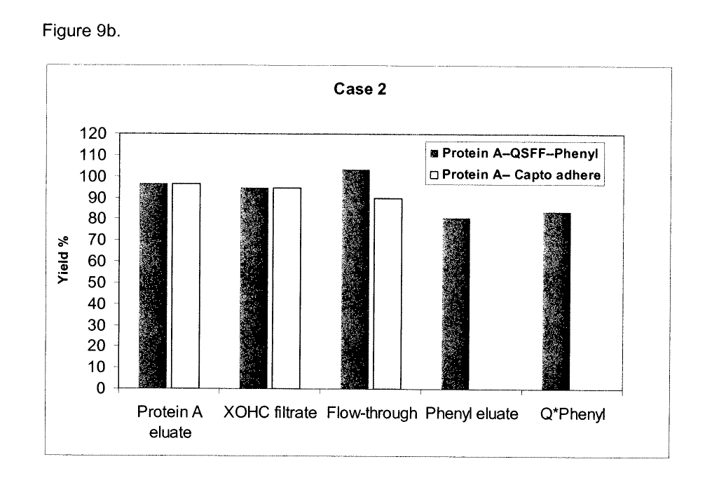

In Case 1, where two XOHC filters were assembled in series and each filter was loaded to 100 L/m² (so the average load based on total filter area is 50 L/m²), nearly all HCPs were removed, with residual HCP levels of from about 1.8 to about 2.4 ng/mg (shown in figures as XOHC filtrate). In addition, about 65% of the leached protein A and about 54% of the aggregates were removed. Host cell DNA was also removed from the product pool to levels below detection. In Case 2, only one XOHC filter was used and loaded to 60 L/m². This resulted in somewhat higher impurity levels: about 56 ng/mg HCP, about 7.2 to 8.6 ng/mg protein A, about 1.8% to 2.0% of aggregates, and about 30 to 40 pg/mg of DNA. Despite the differences in the impurity levels, both XOHC filtrates were purified to yield acceptable final product quality when processing through the subsequent chromatography steps, either by the standard Q plus phenyl columns (standard three-column process) or by the Capto® adhere column (two-column process) (shown in figures as flow through). The Capto® adhere flow-through pool contained less than 4 ng/mg of HCP, which is within the typical acceptable limit (<10 ng/mg). This step appeared to provide more effective protein A clearance than both the Q and phenyl columns and the residual protein A levels were less than 1 ng/mg. In addition, the final product aggregate levels from both processes were comparable, less than 1%, and DNA was below the quantitation limit. FIGS. 8a and 8b summarize the product yields for each purification step. Like most other unit operations, the two-column process gives a step yield of 90%, similar to the combined yield of the Q and phenyl operation, thus making the overall processing yields for both processes comparable.

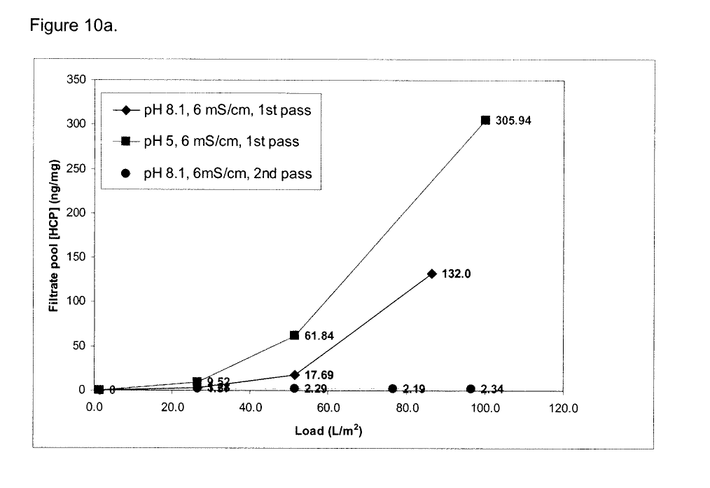

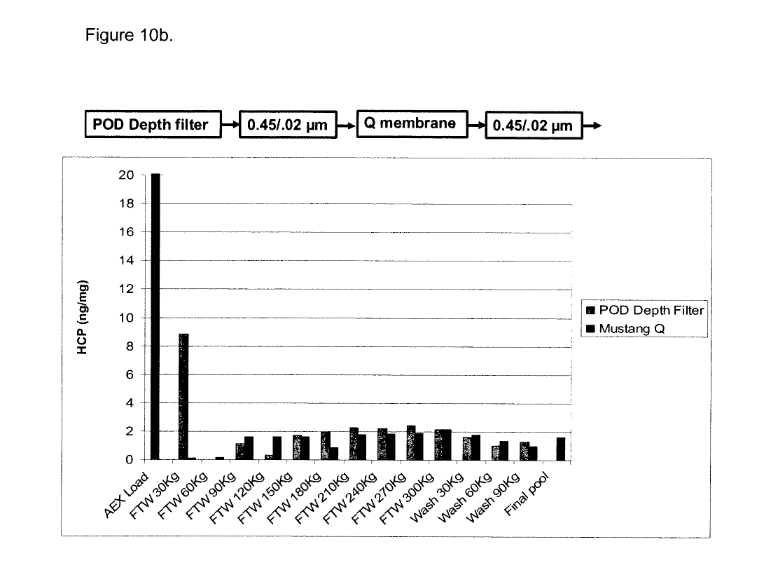

Using a high performance depth filter, for example Millistak+® Pod XOHC depth filter system, with positive charge functionality in a two-column process enhances the robustness of the impurity clearance without significantly affecting product yield. FIG. 10a shows the HCP levels in the filtrate of protein A eluate pool through an XOHC depth filter at different feed loading conditions. Higher pH and lower load level give better HCP clearance. Also, a second pass of filtrate through another XOHC filter results in almost complete clearance of HCP without further column purification. Similar trends were also observed in Cases 1 and 2 as illustrated in FIGS. 5-8. Hence, adequate sizing of the depth filter prior to the mixed-mode intermediate/polishing step ensures robust clearance of product- and process-related impurities throughout the process and consistent production of quality material.

FIG. 10b illustrates the application of the XOHC depth filter to post-Protein A capture/pH inactivated material at a 3000L manufacturing scale. The feedstock was adjusted to pH 7.9 and 5.4 mS/cm conductivity and loaded at 49 L/m² depth filter area. Samples taken during filtration show a greater than 500-fold removal of residual HCP from the feedstock prior to filtration across a Q membrane device.

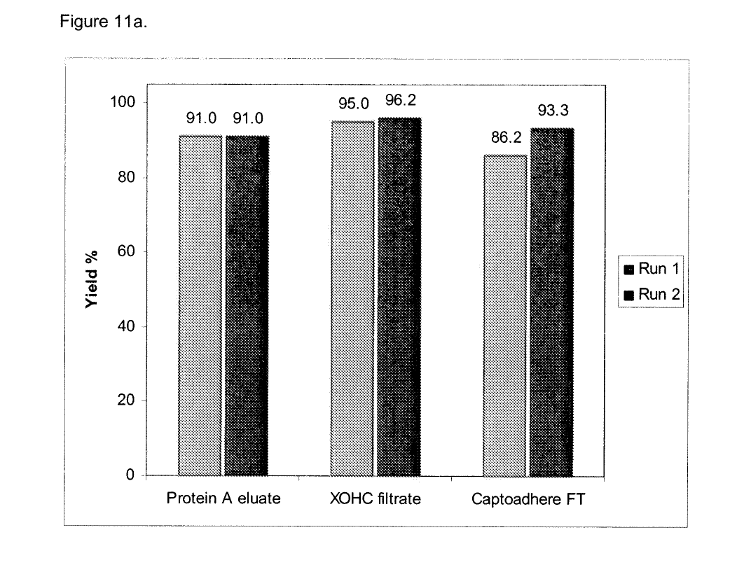

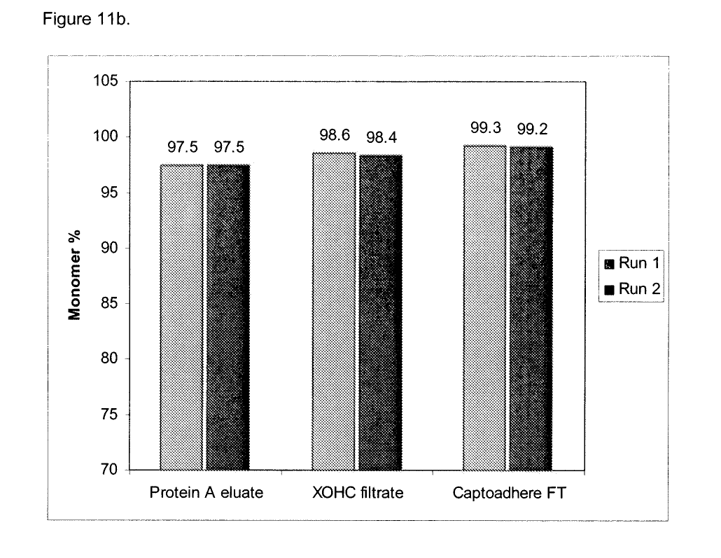

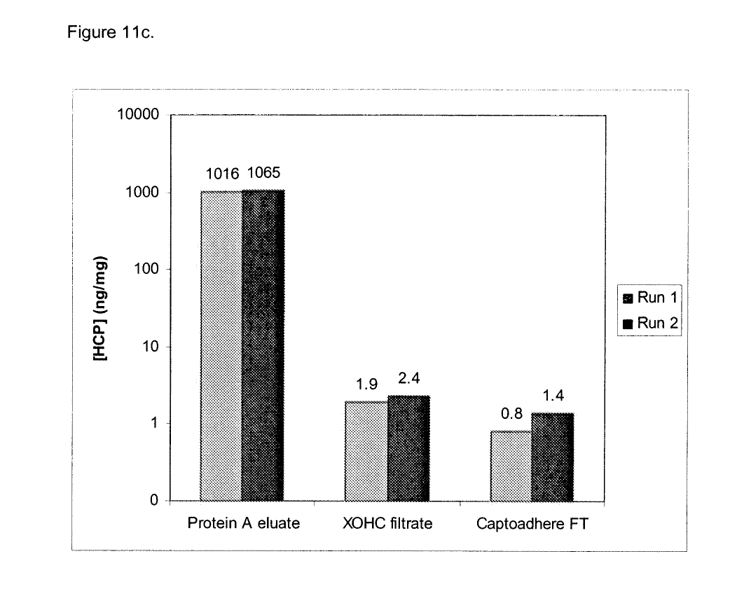

To assess the general applicability of the two-column process for different MAb molecules, the inventors also evaluated PAE1 of MAb B under aforementioned processing conditions. As shown in FIGS. 11a and 11b, the overall process yield and final product purity were similar to that obtained for CH of MAb A, and were also comparable to what was observed in the standard three-column process for this molecule. Hence, this process has the potential to become a platform technology for large-scale purification of monoclonal antibody.

By using a high-performance protein A resin and integrating depth filtration with mixed-mode flow-through operations, the present two-column process can provide yield and product purity equivalent to the standard three-column process. A separate detergent inactivation step used prior to protein A capture can provide additional viral clearance for this process. Moreover, this process eliminates the need for using ammonium sulfate salt, reduces the amount of hardware, tankage, column packing, cleaning, and validation, significantly reduces batch processing time, and ultimately improves process economics.

### Example 2

In this example, a MabSelect™ protein A eluate (herein designated “PAE2”) of MAb A was pH inactivated, neutralized to pH 8 with 1M Tris, pH 9.5 buffer, and then filtered through CUNO 60/90 ZA and delipid depth filter train each followed by a Sartopore 2 0.45/0.22 um sterile filter. The filtrate was then adjusted with 5M NaOH to pH 9.5 and diluted with water to a conductivity range of 6-7 mS/cm. This filtrate contained approximately 3% aggregates, 15 ng/mg HCP, and <1 ng/mg protein A. To better assess the protein A clearance, the sample was spiked with an additional 20 ng/mg of MabSelect™ protein A before being loaded to a 5 mL Capto® adhere column. Two runs were conducted at room temperature, and the specific conditions are summarized in Table 2. The elution pool was analyzed for yield, HCP, protein A, and aggregate/fragment levels.

TABLE 2
Experimental conditions for bind-elute operation on Capto ® adhere column for PAE2 of MAb A.

| Run | | | | | | Pooling |
|---|---|---|---|---|---|---|
| No. | Equilibration | Load | Wash | Elution | Cleaning | criteria |
| 1 | 20 mM Tris, | pH 9.5, 6.8 | Buffer A, | Linear pH gradient | 1M NaOH, | 900 to |
| | 20 mM | mS/cm, titer | 5 CV, 5 | from buffer A to | 5 CV, 1 | 240 mAU |
| | NaCitrate, 20 | 4.9 mg/ml, | min RT | buffer B (20 mM | min RT | during |
| | mM NaCl, pH | load 45 | | Tris, 20 mM | | elution |
| | 9.5, 6.5 | mg/ml at 5 | | NaCitrate, 20 mM | | |
| | mS/cm | min | | NaCl, pH 4, 6.5 | | |
| | (buffer A), 5 | | | mS/cm) in 20 CV, 5 | | |
| | CV, 1 min RT | | | min RT | | |
| 2 | 20 mM Tris, | pH 9.5, 6.8 | Buffer A, | Linear pH gradient | Milli-Q | 200 to |
| | 20 mM | mS/cm, titer | 10 CV, 1 | from buffer A to | water, 5 | 200 mAU |
| | NaCitrate, 20 | 4.98 mg/ml | min RT | buffer B (20 mM | CV, 5 min | during |
| | mM NaCl, pH | load 50 | | Tris, 20 mM | RT; 1M | elution |
| | 9.5, 6.5 | mg/ml at 5 | | NaCitrate, 20 mM | NaOH, 10 | |
| | mS/cm | min | | NaCl, pH 4, 6.5 | CV, 5 min | |
| | (buffer A), 5 | | | mS/cm) in 20 CV, | RT, | |
| | CV, 1 min RT | | | continue buffer B | reverse | |
| | | | | flow for 5 CV 5 min | flow | |
| | | | | RT | | |

Table 3 summarizes the purification performance of the inventive process utilizing a Capto® adhere column in bind-elute mode for PAE2. The impurity levels are comparable to those obtained by a standard three-column process. While the yield was slightly lower in this two-column process as compared to a standard three-column process, the performance of this two-column process was within the acceptable range and can be further optimized, thereby increasing the step yield without compromising the product purity.

TABLE 3
Summary of bind-elute purification performance of Capto ® adhere
column for PAE2 of MAb A.

| | | | | High molecular |
|---|---|---|---|---|
| Run | Yield | HCP | Protein A | weight & low |
| No. | (%) | (ng/mg) | (ng/mg) | molecular weight (%) |
| 1 | 76.6 | 0.79 | Not | 0.74 |
| | | | Determined | |
| 2 | 68.0 | 0.07 | 0 | 0.86 |

### Example 3

Another set of purification experiments were carried out with a process consisting of a Protein A capture, low pH inactivation, XOHC depth filtration and an anion-exchange membrane for final polishing. Again, the CH for MAb A was used in this study and two runs were conducted at different load levels for the XOHC depth filter (Case 1 and Case 2). The protein A capture, pH inactivation and XOHC filtration steps were operated in the same fashion as shown in Example 1. However, the Phenyl column was removed from this process, and the QSFF column was replaced with a 0.08 ml ChromaSorb® membrane device (Millipore Corporation) which was also run in flow-through mode. The ChromaSorb device was wet and cleaned according to manufacturer's protocol, equilibrated with 25 mM Tris buffer with 50 mM NaCl at pH 8, and then challenged with the incoming feed material at 3 kg/L load and 1 ml/min flow rate. After load, the device was washed with the equilibration buffer at the same flow rate. The flow-through fractions were pooled from 200 mAU (UV280) at load to 200 mAU at wash. Key impurities such as HCP, leached protein A, aggregates/fragment and DNA were measured after each step. This process was also compared with the standard three-column process (as detailed in Example 1) for yield and purity.

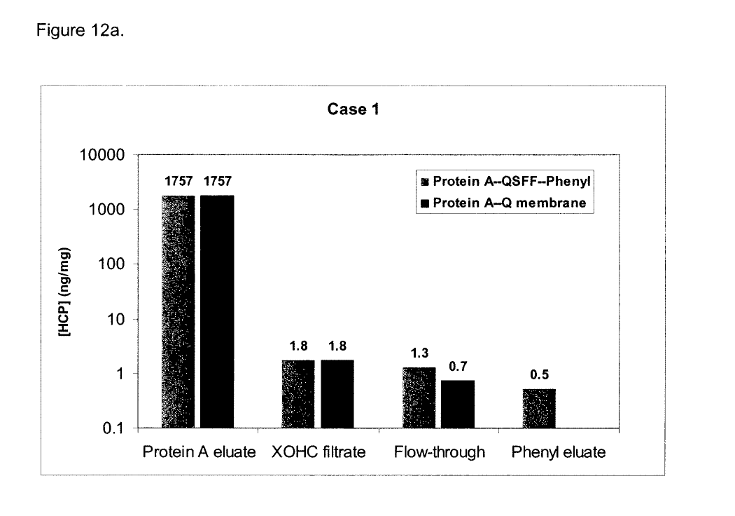

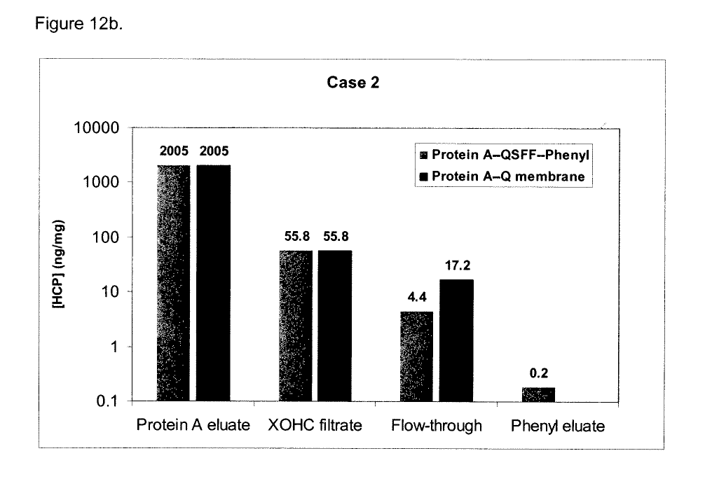

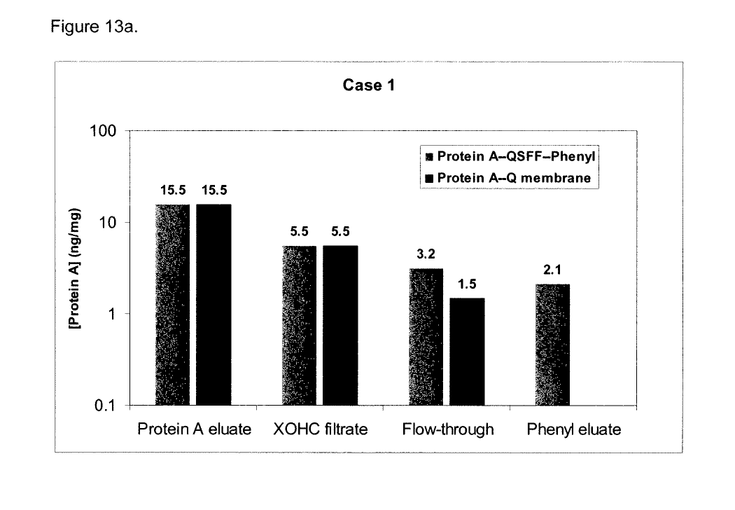

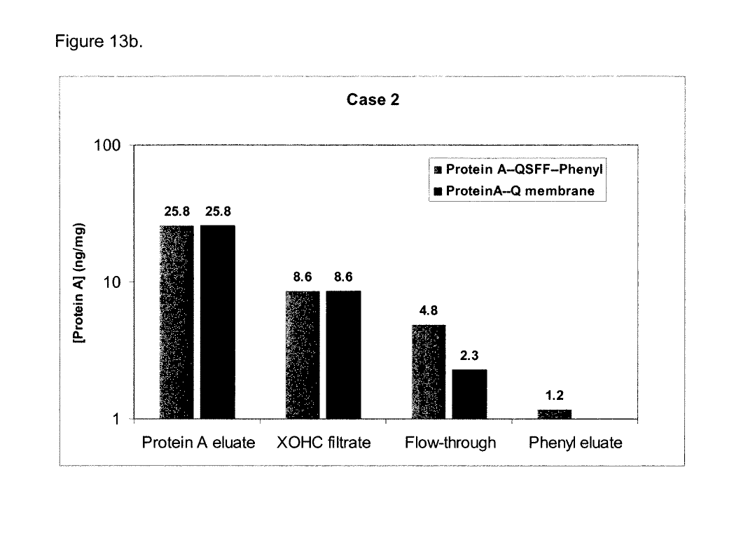

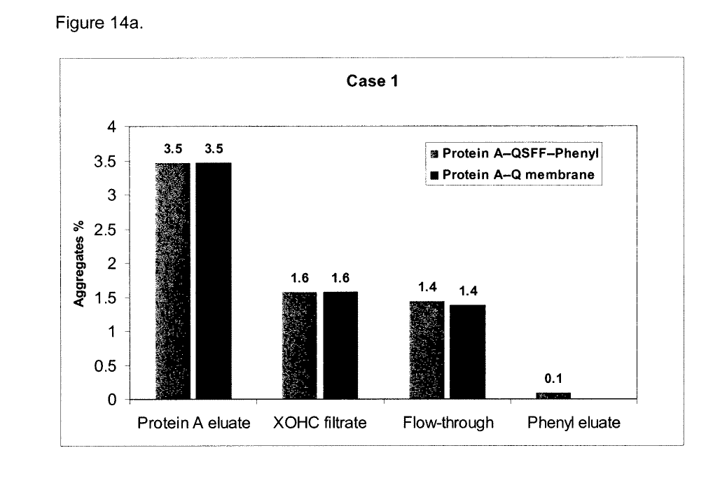

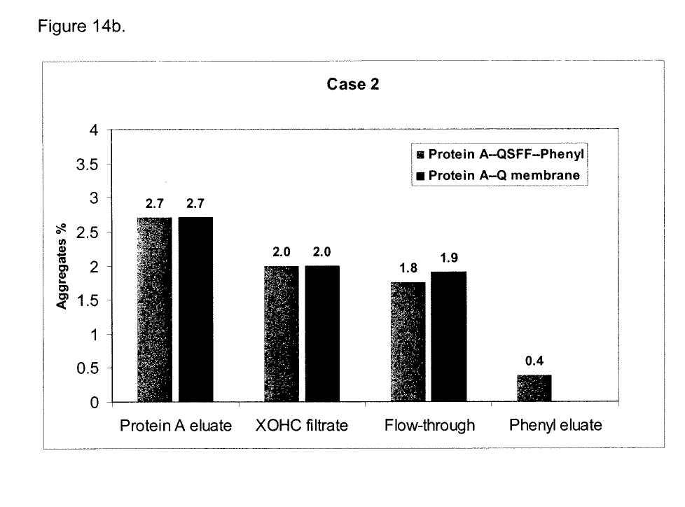

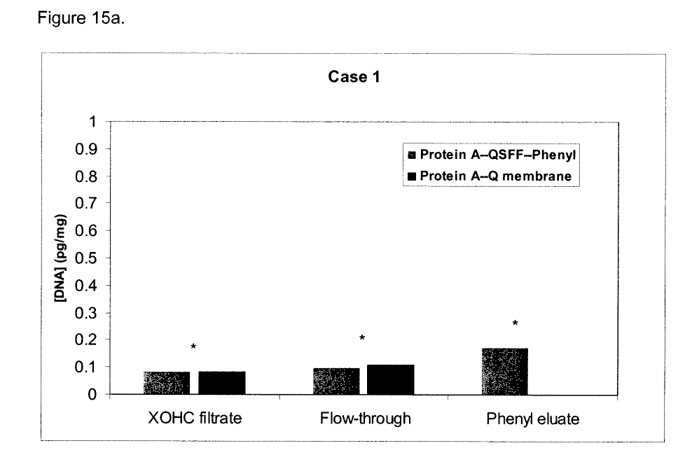

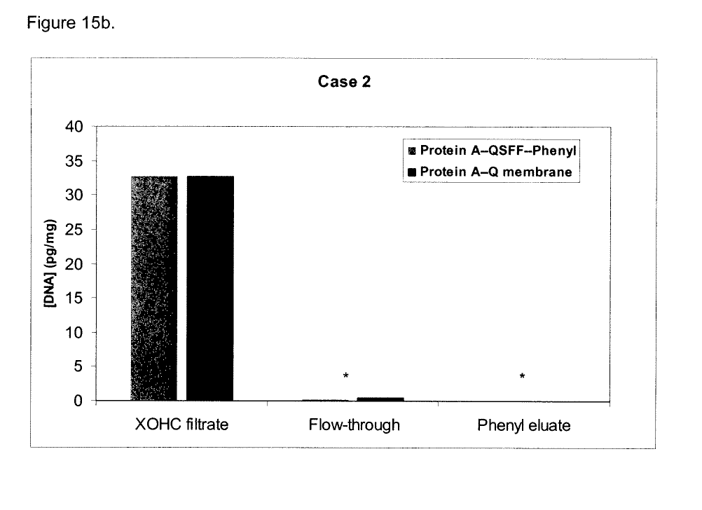

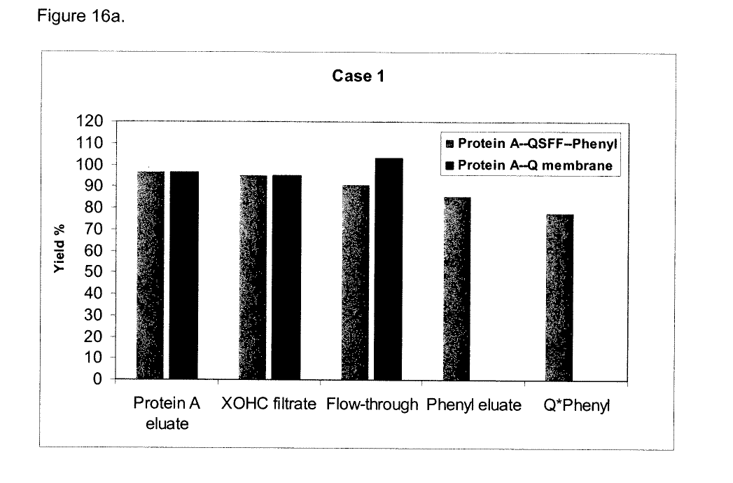

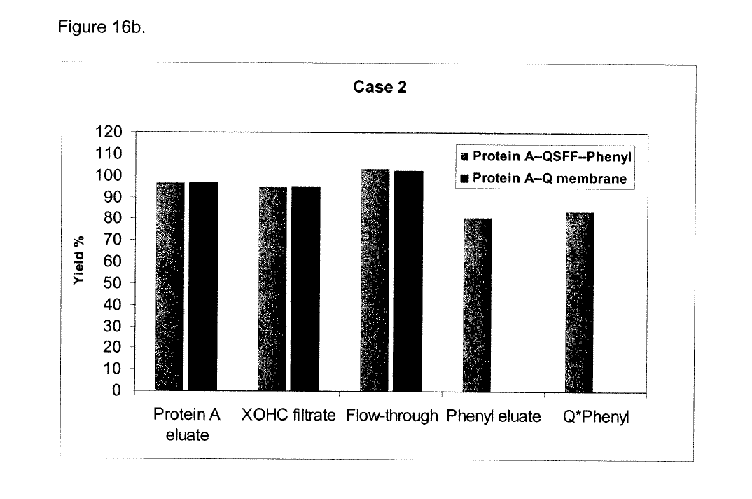

FIGS. 12-15 illustrate impurity profiles for each unit operation in the one-column versus the three-column process. As discussed earlier, when relatively lower feed load was applied to the XOHC depth filter (Case 1), the HCP, aggregates, leached protein A and DNA were more effectively reduced, resulting in very low residual impurity levels. When such POD filtrate was further processed through the Q membrane, all the impurities were further cleared to acceptable levels. For instance, the Q membrane filtrate in Case 1 contained about 0.7 ng/mg HCP, 1.5 ng/mg protein A, 1.4% aggregates and DNA of below quantitation limit. Although the aggregate level was slightly higher than that seen in the phenyl eluate, it could be further minimized by optimizing the process conditions for the Q membrane including pH, conductivity and load level. Alternatively, by sizing up the depth filter prior to the Q membrane step, impurity levels could be lowered from those observed here. As shown in FIG. 16, the step yield for the Q membrane flow-through was comparable to that for the Q column; thus, eliminating the Phenyl column not only reduced the total processing time but also increased the overall purification yield over for the two-column process.

All references cited in this specification, including without limitation, all papers, publications, patents, patent applications, presentations, texts, reports, manuscripts, brochures, books, internet postings, journal articles, and/or periodicals are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the versions contained therein.

What is claimed is:

1. An apparatus for purifying a protein from a sample containing the protein to be purified, comprising:
a. a capture chromatography resin;
b. a depth filter arranged with respect to the capture chromatography resin so that the sample processes through the capture chromatography resin to and through the depth filter; and
c. a mixed-mode chromatography resin arranged with respect to the depth filter so that the sample processes through the depth filter to and through the mixed-mode chromatography resin.

2. The apparatus of claim 1 wherein the capture chromatography resin is selected from the group consisting of an affinity resin, an ion exchange resin, and a hydrophobic interaction resin.

3. The apparatus of claim 1 wherein the capture chromatography resin is selected from the group consisting of a protein A resin, a protein G resin, a protein A/G resin, and a protein L resin.

4. The apparatus of claim 1 wherein the capture chromatography resin and/or mixed-mode chromatography resin is contained within a chromatography column.

5. The apparatus of claim 1 additionally comprising one or more clarification devices for clarifying the protein, arranged to receive the sample before the sample processes to the capture chromatography resin.

6. The apparatus of claim 5 wherein the clarification device is selected from one or more of the group consisting of a centrifuge, a microfilter, an ultrafilter, and a depth filter.

7. The apparatus of claim 1 further comprising a second depth filter arranged to receive the sample from the first depth filter before the sample is processed through the mixed-mode chromatography resin.

8. The apparatus of claim 1 further comprising a sterile filter arranged to receive the sample from the depth filter before the sample is processed through the mixed-mode chromatography resin.

9. The apparatus of claim 1 wherein the mixed-mode chromatography resin comprises a chromatography resin utilizing one or more chromatography mechanisms selected from the group consisting of anion exchange, cation exchange, hydrophobic interaction, hydrophilic interaction, hydrogen bonding, pi-pi bonding, and metal affinity.

10. The apparatus of claim 1 wherein the mixed-mode chromatography resin comprises a chromatography resin utilizing a combination of anion exchange and hydrophobic interaction chromatography mechanisms.

11. An apparatus for purifying a protein from a sample containing the protein to be purified, comprising:
a. a capture chromatography resin;
b. a depth filter arranged with respect to the capture chromatography resin so that the sample processes through the capture chromatography resin to and through the depth filter; and
c. a membrane adsorber arranged with respect to the depth filter so that the sample processes through the depth filter to and through the membrane adsorber.

12. The apparatus of claim 11 wherein the capture chromatography resin is selected from the group consisting of a protein A resin, a protein G resin, a protein A/G resin, and a protein L resin.

13. The apparatus of claim 11 additionally comprising one or more clarification devices for clarifying the protein, arranged to receive the sample before the sample processes to the capture chromatography resin.

14. The apparatus of claim 13 wherein the clarification device is selected from one or more of the group consisting of a centrifuge, a microfilter, an ultrafilter, and a depth filter.

15. The apparatus of claim 11 further comprising a second depth filter arranged to receive the sample from the first depth filter before the sample is processed through the membrane adsorber.

16. The apparatus of claim 11 further comprising a sterile filter arranged to receive the sample from the depth filter before the sample is processed through the membrane adsorber.

17. The apparatus of claim 11 wherein the membrane adsorber is selected from the group consisting of a membrane ion-exchanger, mixed mode ligand membrane and hydrophobic membrane.

18. The apparatus of claim 11 further comprising a pre-bottling filter arranged with respect to the membrane adsorber so that the sample processes through the membrane adsorber to and through the filter.

19. The apparatus of 18 wherein the pre-bottling filter is selected from the group consisting of a viral filter, nanofilter, ultrafilter, and diafilter.

20. An apparatus for purifying a protein from a sample containing the protein to be purified, comprising:
a. a capture chromatography resin;
b. a depth filter arranged with respect to the capture chromatography resin so that the sample processes through the capture chromatography resin to and through the depth filter; and
c. a monolith arranged with respect to the depth filter so that the sample processes through the depth filter to and through the monolith.

21. A method for purifying a protein comprising:
a. providing a sample containing the protein;
b. processing the sample through a capture chromatography resin to provide a first eluate comprising the protein;
c. after the sample is processed through the capture chromatography resin, processing the first eluate through a depth filter to provide a filtered eluate comprising the protein; and
d. after the first eluate is processed through the depth filter, processing the filtered eluate through a mixed-mode chromatography resin to provide a second eluate comprising the protein.

22. The method of claim 21 wherein the capture chromatography resin is selected from the group consisting of an affinity resin, an ion exchange resin, and a hydrophobic interaction resin.

23. The method of claim 21 wherein the capture chromatography resin is selected from the group consisting of a protein A resin, a protein G resin, a protein A/G resin, and a protein L resin.

24. The method of claim 21 wherein the protein is selected from the group consisting of a protein fragment, an antibody, a monoclonal antibody, an immunoglobulin, and a fusion protein.

25. The method of claim 21 wherein the sample is a cell culture.

26. The method of claim 21 wherein the sample is clarified prior to processing through the capture chromatography resin.

27. The method of claim 26 wherein the sample is clarified by a clarification method selected from the group consisting of centrifugation, microfiltration, ultrafiltration, depth filtration, sterile filtration, and treatment with a detergent.

28. The method of claim 21 wherein the first eluate is subjected to viral inactivation after processing through the capture chromatography resin but before processing through the depth filter.

29. The method of claim 28 wherein the viral inactivation comprises a method selected from the group consisting of treatment with acid, detergent, chemicals, nucleic acid cross-linking agents, ultraviolet light, gamma radiation, and heat.

30. The method of claim 21 wherein the filtered eluate is processed through a depth filter a second time.

31. The method of claim 21 wherein the mixed-mode chromatography resin comprises a chromatography resin utilizing one or more chromatography techniques selected from the group consisting of anion exchange, cation exchange, hydrophobic interaction, hydrophilic interaction, hydrogen bonding, pi-pi bonding, and metal affinity.

32. The method of claim 21 wherein the mixed-mode chromatography resin comprises a chromatography resin utilizing a combination of anion exchange and hydrophobic interaction chromatography techniques.

33. The method of claim 21 wherein, after processing through the mixed-mode chromatography resin, the second eluate is subjected to further filtration.

34. The method of claim 33 wherein the further filtration comprises one or more of the methods selected from the group consisting of viral filtration, nanofiltration, ultrafiltration, and diafiltration.

35. The method of claim 21 wherein filtered eluate is processed through the mixed-mode chromatography resin in flow-through mode.

36. The method of claim 21 wherein filtered eluate is processed through the mixed-mode chromatography resin in bind-elute mode.

37. A method for purifying a protein comprising:
a. providing a sample containing the protein;
b. processing the sample through a capture chromatography resin to provide a first eluate comprising the protein;
c. after the sample is processed through the capture chromatography resin, processing the first eluate through a depth filter to provide a filtered eluate comprising the protein; and
d. after the first eluate is processed through the depth filter, processing the filtered eluate through a membrane adsorber to provide a second eluate comprising the protein.

38. A method for purifying a protein comprising:
a. providing a sample containing the protein;
b. processing the sample through a capture chromatography resin to provide a first eluate comprising the protein;
c. after the sample is processed through the capture chromatography resin, processing the first eluate through a depth filter to provide a filtered eluate comprising the protein; and
d. after the first eluate is processed through the depth filter, processing the filtered eluate through a monolith to provide a second eluate comprising the protein.
